(19) United States
(12) Patent Application Publication
(10) Pub. No.: US 2023/0048219 A1
(43) Pub. Date: Feb. 16, 2023

(54) PROCESS FOR CONVERTING AMIDE TO AMINE

(71) Applicant: RHODIA OPERATIONS, Lyon (FR)

(72) Inventors: Willinton Yesid Hernandez Enciso, Shanghai (CN); Stephane Streiff, Lyon (FR)

(73) Assignee: RHODIA OPERATIONS, Lyon (FR)

(21) Appl. No.: 17/782,978

(22) PCT Filed: Dec. 6, 2019
(86) PCT No.: PCT/CN2019/123579

§ 371 Date: Jun. 6, 2022

(51) Int. Cl. C07C 211/07; C07C 211/08; C07C 209/50; B01J 21/04; B01J 21/08; B01J 23/22; B01J 23/46

(52) U.S. Cl. CPC C07C 211/07; C07C 211/08; C07C 209/50; B01J 21/04; B01J 21/08; B01J 23/22; B01J 23/46

(57) ABSTRACT

The present invention relates to a process for converting an amide into an amine comprising hydrogenation of the amide at a temperature not higher than 130° C. and a hydrogen pressure not higher than 50 bar in the presence of a supported heterogeneous catalyst preparable by a method comprising depositing vanadium on a supported noble metal catalyst by impregnation.

### TECHNICAL FIELD

The present invention relates to a process for preparing an amine. In particular, the present invention relates to a process for converting an amide into an amine.

### BACKGROUND ART

Amines constitute an important class of compounds with extensive use as medicines or basic raw materials for the preparation of pharmaceuticals. Therefore, economically viable and green methods of synthesizing amine are important. A simple and direct approach would be catalytic reduction of amides.

The efficient hydrogenation of amides is a highly desirable synthesis route for the sustainable production of amines at a large scale. Nevertheless, due to the high stability of the carboxamide function (amides have relatively low electrophilicity of the C═O group), the utilization of heterogeneous hydrogenation catalysts for this reaction is also accompanied by the need of rather harsh reaction conditions (e.g. H₂ pressure over 200 bar and reaction temperature over 250° C.). Reference may be made to, for example, Schneider, H. J. et al., Journal of the American Chemical Society 1952, 74, 4287; and Wojcik, B. et al., Journal of the American Chemical Society 1934, 56, 2419.

The more recent developments on this challenging topic point out to the necessity of using bifunctional catalysts (bimetallic or multimetallic) characterized by an optimized synergistic interaction between the active sites. Thus, it is well accepted that the combination of hydrogenation active sites (e.g. noble metal nanoparticles such as, Pt, Rh, Pd) and oxophilic sites (and/or Lewis-acid sites, typically Rhenium and Molybdenum-based) can favour the hydrogenation process at relatively mild reaction conditions (T≤150° C. and P_{H2}≤50 bar). Moreover, those bifunctional systems have shown to be effective for the transformation of a broad range of substrates, especially tertiary amides (mainly cyclic amides, such as N-acetylpiperidine) and secondary amides. Reference may be made to, for example, Whyman R. et al., Selective hydrogenation of amides using bimetallic Ru/Re and Rh/Re catalysts, J. Catal., 278, (2011), 228; and Shimizu K. et al., Lewis Acid-Promoted Heterogeneous Platinum Catalysts for Hydrogenation of Amides to Amines, Chem. Select, 1, (2016), 736.

US 2010179349 discloses a process for producing a tertiary amine by reducing an amide compound in the presence of a catalyst containing a sponge copper catalyst obtained by leaching alloy particles containing copper and aluminum and drying the thus leached alloy particles. This patent application also discloses a process for producing high-purity aliphatic tertiary amines containing a less amount of by products at a high yield by subjecting aliphatic acid amides to hydrogenation reduction under solvent-free moderate conditions. In particular, the process disclosed in this patent application includes the step of (a) reducing the amide compound in the presence of a sponge copper catalyst obtained under solvent-free condition at a temperature from 140° C. to about 300° C., preferably from 160° C. to 280° C., and still more preferably from 180° C. to 270° C., a reaction pressure from normal pressure to about 25 MPaG. And from the view point of enhancing the purity of the tertiary amine obtained in the step (a) with a still higher selectivity, it is preferred that a dialkyl amine containing a linear or branched alkyl group having 1 to 6 carbon atoms and hydrogen are further introduced into the reaction system in the presence of a catalyst, which can be the same used in step (a).

WO 2005066112 discloses a method for catalytic reduction of an amide for the preparation of an amine at a temperature of below 200° C. and a pressure of below 50 bar, the catalyst being chosen from bimetallic and trimetallic catalysts of the group consisting of ABC, AB, AC and BC, wherein A is a metal chosen from the group consisting of Co, Fe, Ir, Pt, Rh and Ru, B is a metal chosen from the group consisting of Cr, Mo, Re and V, and, C is a metal chosen from the group consisting of Cu, In and Zn. The two or three metals forming the catalysts were deposited onto the carrier by incipient wetness impregnation directly from aqueous solutions containing a mixture of all desired metal salts.

Besides the catalytic system, the reaction conditions needed for the efficient hydrogenation also depend on the type of amide to be reduced. For instance, primary amides normally require higher reaction temperatures and H₂ pressure, as compared with secondary and tertiary ones. Long-chain aliphatic amides are also challenging substrates to be hydrogenated since those molecules are highly susceptible to other side reaction involving the breaking of C—C and C—N bonds.

Thus there is still a need for a process for hydrogenation of amides to corresponding amines at mild conditions.

### SUMMARY OF THE INVENTION

An object of the present invention is to provide a process for hydrogenation of amides to corresponding amines at mild conditions.

Thus, according to a first aspect, the present invention provides a process for converting an amide into an amine comprising hydrogenation of the amide at a temperature not higher than 130° C. and a hydrogen pressure not higher than 50 bar in the presence of a supported heterogeneous catalyst preparable by a method comprising depositing vanadium on a supported noble metal catalyst by impregnation.

According to a second aspect, the present invention provides a converting an amide into an amine comprising the steps of:

(i) preparing a supported heterogeneous catalyst by a method comprising depositing vanadium on a supported noble metal catalyst by impregnation, and

(ii) causing hydrogenation of the amide at a temperature not higher than 130° C. and a hydrogen pressure not higher than 50 bar in the presence of the so-prepared supported heterogeneous catalyst to obtain the amine.

With the process according to the present invention, an amide can be converted a corresponding amine at a relatively higher selectivity at neat reaction conditions, and even up to 100% at diluted reaction conditions.

According to a third aspect, the present invention provides a mixture comprising a first amine of formula (II) and an alcohol of formula (III):

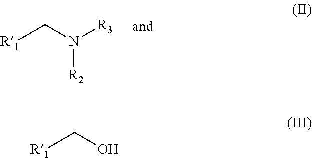

wherein:

R′₁ is cyclohexyl which is optionally substituted by a linear or branched C1-C4 alkyl;

R₂ and R₃, independently from each other, are hydrogen, or linear or branched C1-C4 alkyl;

the molar ratio of the first amine to the alcohol is greater than 2.5, preferably greater than 3.

Other subjects and characteristics, aspects and advantages of the present invention will emerge even more clearly on reading the detailed description and the examples that follow.

### BRIEF DESCRIPTION OF THE DRAWINGS

A more particular description of the present invention will be rendered by reference to the appended drawings, in which:

FIG. 1 shows H₂-TPR profiles for the catalysts prepared in Examples 8-9 and Comparative Example 2;

FIG. 2 shows CO-chemisorption results for the catalysts prepared in Examples 8-9 and Comparative Example 2.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Should the disclosure of any patents, patent applications and publications which are incorporated herein by reference conflict with the description of the present application in the extent that it may render a term unclear, the present description shall take precedence.

Unless otherwise specified, all numerical values expressing amount of ingredients, reaction conditions and the like used in the description and claims are to be understood as being modified by the term “about”. Accordingly, unless indicated to the contrary, the numerical values and parameters described herein are approximate values which are capable of being changed according to the desired performance obtained as required.

As used herein, the term “supported heterogeneous catalyst” means a catalyst comprising a noble metal and vanadium on a support according to the present invention.

As used herein, the term “supported noble metal catalyst” means a catalyst comprising only a noble metal on a support.

Converting an Amide into an Amine

According to a first aspect, the present invention provides a process for converting an amide into an amine comprising hydrogenation of the amide at a temperature not higher than 130° C. and a hydrogen pressure not higher than 50 bar in the presence of a supported heterogeneous catalyst preparable by a method comprising depositing vanadium on a supported noble metal catalyst by impregnation.

In some embodiments, wherein the amide is of formula (I) and the amine is of formula (II),

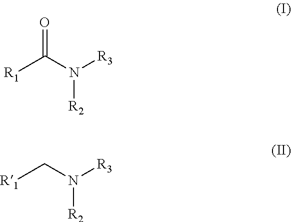

wherein

R₁ is a group selected from linear or branched C1-C20 alkyl, and phenyl which is optionally substituted by a linear or branched C1-C4 alkyl,

R′₁ is identical to R₁ when R₁ is a linear or branched C1-C20 alkyl and R′₁ is cyclohexyl which is optionally substituted by a linear or branched C1-C4 alkyl when R₁ is phenyl which is optionally substituted by a linear or branched C1-C4 alkyl,

R₂ and R₃, independently from each other, are hydrogen, or linear or branched C1-C4 alkyl, or

R₂ and R₃ together with the nitrogen atom they attached to form a piperidine ring which is optionally substituted by a linear or branched C1-C4 alkyl.

In some embodiments, R₁ represents a linear or branched C1-C14 alkyl, or phenyl which is optionally substituted by a linear or branched C1-C4 alkyl.

For example, the amide of formula (I) is selected from N,N-dimethyl lauryl amide, benzamide, lauramide and 1-acetyl piperidine.

Preferably, the hydrogenation is carried out at a temperature from 70° C. to 130° C. and a hydrogen pressure from 10 to 50 bar.

More preferably, the hydrogenation is carried out at a temperature from 100° C. to 130° C. and a hydrogen pressure from 30 to 50 bar.

The hydrogenation can be carried out under diluted or neat condition.

For example, in an embodiment, the hydrogenation is carried out for diluted amide in a solvent such as dimethoxy ethane, for example, at a concentration ranging from 2 wt. % to 50 wt. %, for example, 2.5 wt. %.

As examples of the noble metal can be used in the supported heterogeneous catalyst, mention can be made to rhodium(Rh), platinum (Pt), ruthenium (Ru), and iridium (Ir).

In a preferred embodiment, Rh is used as the noble metal.

Advantageously, the noble metal is present in amount from 1 wt. % to 10 wt. %, preferably 2 wt. % to 8 wt. %, more preferably 3 wt. % to 7 wt. % in the supported heterogeneous catalyst, relative to the total weight of the supported heterogeneous catalyst.

Advantageously, vanadium is present in amount from 0.5 wt. % to 10 wt. %, preferably 1 wt. % to 8 wt. %, more preferably 2 wt. % to 7 wt. % in the supported heterogeneous catalyst, relative to the total weight of the supported heterogeneous catalyst.

Vanadium exists in the form of V_{y}Oₓ (V^{δ+}=5⁺, 4⁺) in the supported heterogeneous catalyst according to the present invention.

Advantageously, the molar ratio of the noble metal to vanadium is from 0.5 to 10, preferably from 1 to 2.

In some embodiments, the molar ratio of the noble metal to vanadium is 1:1.

In some embodiments, the molar ratio of the noble metal to vanadium is 1:0.5.

The support for the supported heterogeneous catalyst can be selected from alumina (Al₂O₃), silica (SiO₂) and activated carbon (C).

Preferably, the support has a specific surface area of over 50 m²/g, preferably from 50 m²/g to 800 m²/g and more preferably 100 m²/g to 300 m²/g.

In some embodiments, the support used is alumina (Al₂O₃), for example γ-Al₂O₃. The supported heterogeneous catalyst is characterized by the presence of a reduction peak at a temperature below 200° C., preferably at a temperature from 40° C. to 130° C., more preferably from 50° C. to 100° C., still more preferably from 55° C. to 90° C., as determined by H₂-TPR analysis.

The supported heterogeneous catalyst is characterized by the presence of a hydrogen consumption of at least 0.5 mmol H₂/g, preferably from 0.7 to 0.9 mmol H₂/g at one or more temperature(s) in the reduction from 40° C. to 200° C., as determined by H₂-TPR analysis.

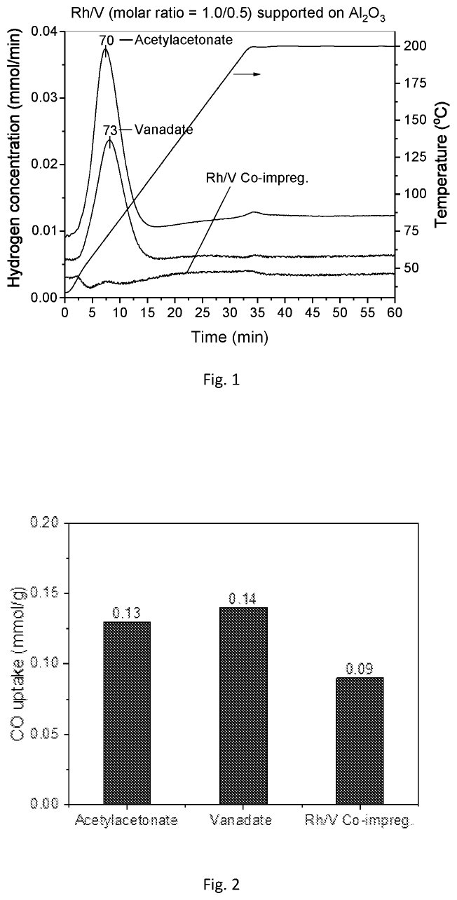

The hydrogen consumption is calculated by integrating the area of the signal (hydrogen concentration, presented as mmol/min), as a function of time (in minutes) as shown in FIG. 1.

The supported heterogeneous catalyst according to the present invention is characterized by a CO uptake of at most 0.12 mmol/g, preferably at most 0.11 mmol/g, more preferably at most 0.10 mmol/g, as determined by CO-chemisorption analysis.

The supported heterogeneous catalyst is characterized by a CO uptake which is at least 10%, preferably at least 20%, more preferably at least 30%, still more preferably at least 40% higher than the CO uptake of a reference catalyst prepared by co-impregnating the same amounts of noble metal and V on a same support using the same impregnation conditions.

The supported heterogeneous catalyst is characterized by the fact that they are free of noble metal-V-type solid solution phase or contain such a phase in an amount that is lower than the amount of this phase which is present in a reference catalyst prepared by co-impregnating the same amounts of noble metal and V on a same support using the same impregnation conditions.

As used therein, H₂-TPR and CO-chemisorption analysis of a catalyst were performed in a Micromeritics AutoChem II 2920 apparatus with a thermal conductivity detector (TCD). For each experiment, approximately 100 mg catalyst was placed in a U-shaped quartz tube (i.d.=10 mm) and reduced in a flow of 10% H₂/Ar while ramping the temperature up to 200° C. at the rate of 10° C./min, and then held at 200° C. for 30 min (this part corresponds to the H2-TPR analysis). After that, the sample was cooled down to 50° C. and flushed with He for 30 min. The loop gas of 10% CO/He was pulsed over the sample and the TCD signal was recorded until the peak area became constant (this part corresponds to the CO-TPD analysis).

Advantageously, the hydrogenation is carried out with the molar ratio of the noble metal in the supported heterogeneous catalyst to the amide from 0.5% to 35%, preferably from 0.8% to 30%.

According to the second aspect, the present invention provides a converting an amide into an amine comprising the steps of:

(i) preparing a supported heterogeneous catalyst by a method comprising depositing vanadium on a supported noble metal catalyst by impregnation, and

(ii) causing hydrogenation of the amide at a temperature not higher than 130 C and a hydrogen pressure not higher than 50 bar in the presence of the so-prepared supported heterogeneous catalyst to obtain the amine.

In some embodiment, the amide is of formula (I) and the amine is of formula (II),

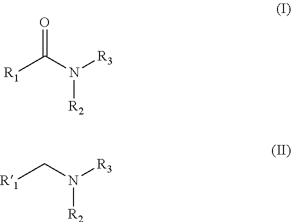

wherein

R₁ is a group selected from linear or branched C1-C20 alkyl, and phenyl which is optionally substituted by a linear or branched C1-C4 alkyl,

R′₁ is identical to R₁ when R₁ is a linear or branched C1-C20 alkyl and R′₁ is cyclohexyl which is optionally substituted by a linear or branched C1-C4 alkyl when R₁ is phenyl which is optionally substituted by a linear or branched C1-C4 alkyl,

R₂ and R₃, independently from each other, are hydrogen, or linear or branched C1-C4 alkyl, or

R₂ and R₃ together with the nitrogen atom they attached to form a piperidine ring which is optionally substituted by a linear or branched C1-C4 alkyl.

In some embodiments, R₁ represents a linear or branched C1-C14 alkyl, or phenyl which is optionally substituted by a linear or branched C1-C4 alkyl.

For example, the amide of formula (I) is selected from N,N-dimethyl lauryl amide, benzamide, lauramide and 1-acetyl piperidine.

In some embodiments, depositing vanadium on the supported noble metal catalyst comprising depositing a vanadium precursor on the supported noble metal catalyst by impregnation, especially wet impregnation.

As examples for the vanadium precursor, mention can be made to Vanadyl (IV) acetylacetonate and ammonium metavanadate.

In some embodiments, depositing vanadium on the supported noble metal catalyst is carried out as follows:

i) dissolving the vanadium precursor in a solvent to obtain a vanadium-containing solution,

ii) adding the supported noble metal catalyst to the vanadium-containing solution to form an uniform mixture;

iii) evaporating the solvent to yield a powder;

iv) drying and calcining the powder to obtain the supported heterogeneous catalyst.

As examples for the solvent, mention can be made to acetone, water, and ethanol.

Advantageously, the dried powder is calcined at a temperature from 300° C. to 400° C. for 4-6 hours.

As examples of the noble metal can be used in the supported heterogeneous catalyst, mention can be made to rhodium(Rh), platinum (Pt), ruthenium (Ru), and iridium (Ir).

In a preferred embodiment, Rh is used as the noble metal.

Advantageously, the noble metal is present in amount from 1 wt. % to 10 wt. %, preferably 2 wt. % to 8 wt. %, more preferably 3 wt. % to 7 wt. % in the supported heterogeneous catalyst, relative to the total weight of the supported heterogeneous catalyst.

Advantageously, vanadium is present in amount from 0.5 wt. % to 10 wt. %, preferably 1 wt. % to 8 wt. %, more preferably 2 wt. % to 7 wt. % in the supported heterogeneous catalyst, relative to the total weight of the supported heterogeneous catalyst.

Vanadium exists in the form of V_{y}Oₓ (V^{δ+}=5⁺, 4⁺) in the supported heterogeneous catalyst according to the present invention.

Advantageously, the molar ratio of the noble metal to vanadium is from 0.5 to 10, preferably from 1 to 2.

In some embodiments, the molar ratio of the noble metal to vanadium is 1:1.

In some embodiments, the molar ratio of the noble metal to vanadium is 1:0.5.

The support for the supported heterogeneous catalyst can be selected from alumina (Al₂O₃), silica (SiO₂) and activated carbon (C).

Preferably, the support has a specific surface area of over 50 m²/g, preferably from 50 m²/g to 800 m²/g and more preferably 100 m²/g to 300 m²/g.

In some embodiments, the support used is alumina (Al₂O₃), for example γ-Al₂O₃.

In a particular embodiment, the supported heterogeneous catalyst is prepared as follows:

i) dissolving vanadyl acetylacetonate (V(acac)₂) in acetone to obtain a vanadium-containing solution;

ii) adding a Rh/Al₂O₃ catalyst to the vanadium-containing solution;

iii) evaporating acetone under a reduced pressure to yield a powder;

iv) drying and calcining the powder under static air at a temperature from 300° C. to 400° C., for example, 300° C. for 3-5 hours, for example, 4 hours to provide the supported heterogeneous catalyst.

In a particular embodiment, the supported heterogeneous catalyst is prepared as follows:

i) dissolving ammonium metavanadate in water to obtain a vanadium-containing solution;

ii) adding a Rh/Al₂O₃ catalyst to the vanadium-containing solution to form an uniform mixture;

iii) evaporating water under a reduced pressure to yield a powder;

iv) drying and calcining the powder under static air at a temperature from 300° C. to 400° C., for example, 300° C. for 3-5 hours, for example, 4 hours to obtain the supported heterogeneous catalyst.

In an illustrated example, the supported heterogeneous catalyst is prepared as follows.

A solution of vanadyl acetylacetonate (V(acac)₂) was prepared by dissolving the desired amount of V(acac)₂) in acetone at room temperature, under stirring for 30 minutes. Then, a Rh/Al₂O₃ catalyst was added to the V(acac)₂)/acetone solution under vigorous stirring, at room temperature, maintaining the stirring for 4 hours. Afterwards, acetone was evaporated under reduced pressure and finally, the recovered powder was dried in oven at 80° C. overnight, and calcined under static air at 300° C. for 4 hours (10° C./min heating ramp).

The supported noble metal catalyst can be commercial available.

As an example for the supported noble metal catalyst useful for the present invention, mention can be made to C301099-5 from Johnson Mattey company, a Rh/Al₂O₃ catalyst containing 5 wt. % Rh, relative to the total weight of the supported rhodium catalyst.

Alternatively, the supported noble metal catalyst can be produced with a conventional method in the art.

For example, the supported noble metal can be produced by depositing a noble metal precursor on the support by impregnation.

Preferably, the hydrogenation is carried out at a temperature from 70° C. to 130° C. and a hydrogen pressure from 10 to 50 bar.

More preferably, the hydrogenation is carried out at a temperature from 100° C. to 130° C. and a hydrogen pressure from 30 to 50 bar.

The hydrogenation can be carried out under diluted or neat condition.

For example, in an embodiment, the hydrogenation is carried out for diluted amide in a solvent such as dimethoxy ethane, for example, at a concentration ranging from 2 wt. % to 50 wt. %, for example, 2.5 wt. %.

Advantageously, the hydrogenation is carried out with the molar ratio of the noble metal in the supported heterogeneous catalyst to the amide from 0.5% to 35%, preferably from 0.8% to 30%.

With the process according to the present invention, an amide can be converted a corresponding amine at a relatively higher selectivity at neat reaction conditions, and even up to 100% at diluted reaction conditions.

According to a third aspect, the present invention provides a mixture comprising a first amine of formula (II) and an alcohol of formula (III):

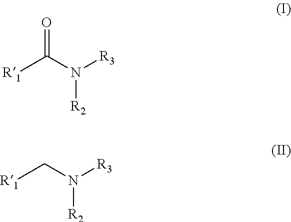

wherein:

R′₁ is cyclohexyl which is optionally substituted by a linear or branched C1-C4 alkyl;

R₂ and R₃, independently from each other, are hydrogen, or linear or branched C1-C4 alkyl;

The molar ratio of the first amine to the alcohol is greater than 2.5, preferably greater than 3.

In some embodiments, R₃ is H.

In some embodiments, the mixture comprises a second amine of formula (IV):

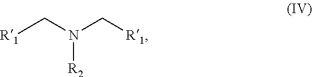

wherein R′₁ and R₂ have the same meaning as defined above.

In some embodiments, R₂ is H.

In some embodiments, the mixture comprises the second amine, the molar ratio of the first amine to the second amine is greater than 5, preferably greater than 7.5.

In some embodiments, R′₁ is cyclohexyl.

The process according to the present invention represents an important advantage for the industrial preparation of amines, as it could simplify the current preparation pathway, going from 3 to 2 steps process, as shown in scheme 1 below, which takes the preparation of N,N-dimethyl fatty amine as an example.

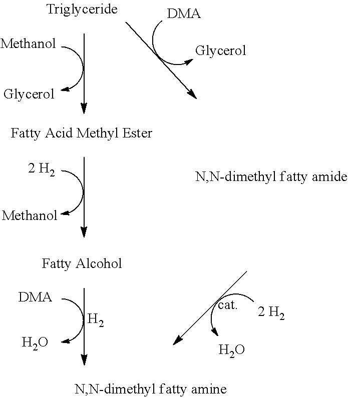

Scheme 1. 3 step process is a current pathway used for the synthesis of aliphatic amines; 2 step process is a proposed reaction pathway using the supported heterogeneous catalyst according to the present invention.

In addition, compared with the process previously reported for this type of reaction, the process according to the present invention shows several advantages, including:

i) high conversion and selectivity after only 1 hour reaction (for example, above 90% conversion and 100% selectivity under diluted conditions);

ii) it does not need the presence of molecular sieve to capture the water formed during the reaction;

iii) it is effective for the hydrogenation of amides, especially primary amides and long-chain aliphatic amides;

iv) the catalyst used therein can be reused several times (up to 5 times) without significant losses in the catalytic efficiency.

Without limited to any specific theory, it is believed that the improved catalytic efficiency is caused by the interaction generated between the noble metal and the deposited vanadium.

### EXAMPLES

The technical features and technical effects of the present invention will be further described below in conjunction with the following examples so that the skilled in the art would fully understand the present invention. It will be readily understood by the skilled in the art that the examples herein are for illustrative purposes only and the scope of the present invention is not limited thereto.

### Example(Ex.) 1: Preparation of Catalysts

V_{y}Oₓ/Rh/A₂O₃ type catalysts were prepared as follows.

A solution of vanadyl acetylacetonate (V(acac)₂) was prepared by dissolving the desired amount of V(acac)₂ in acetone (90 mL) at room temperature, under stirring for 30 minutes. Then, 1 g of Rh/Al₂O₃ catalyst (containing 5 wt. % of Rh, relative to the total weight of the Rh/Al₂O₃ catalyst, from Johnson Matthey) was added to the vanadium solution under vigorous stirring, at room temperature, maintaining the stirring for 4 hours.

Afterwards, the solvent was evaporated under reduced pressure and finally, the recovered powder was dried in oven at 80° C. overnight, and calcined under static air at 300° C. for 4 hours (10° C./min heating ramp).

Based on the amount of vanadyl acetylacetonate used, the V_{y}Oₓ/Rh/Al₂O₃ type catalysts with Rh/V molar ratio of 1/1 and 1/0.5 were obtained.

### Comparative Example (Comp.) 1: Hydrogenation of N,N-Dimethyl Laurylamide

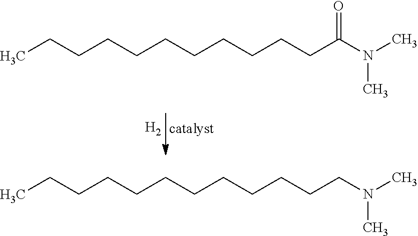

Hydrogenation was performed in a 30 mL Taiatsu autoclave at 130° C. and 30 bar H₂ pressure for 1 hour in the presence of the unmodified catalyst used in Example 1, i.e. a Rh/Al₂O₃ catalyst containing 5 wt. % of Rh from Johnson Matthey. The hydrogenation was carried out under diluted condition using dimethyl ethane (DME) as a solvent. Molecular Sieve (4 Å) was used as water scavenging agent.

In particular, N,N-dimethyl laurylamide in dimethoxy ethane (DME), 5 mL in total, was introduced in the reactor, followed by the addition of 0.15 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard. The filtered samples were analyzed by gas chromatography.

Conversion, yield and selectivity were calculated by GC-analysis, using heptane or dodecane as external standard. Error bar can be considered as ±5%.

The conversion, yield and selectivity were summarized in Table 1.

### Examples (Ex.) 2-6: Hydrogenation of N,N-Dimethyl Laurylamide

Hydrogenation was performed in a 30 mL Taiatsu autoclave at 130° C. and 30 bar H₂ pressure at a given substrate concentration and reaction time, as specified in Table 1 in the presence of a V_{y}Oₓ/Rh/Al₂O₃ type catalyst prepared in Example 1.

The hydrogenation was carried out under diluted condition using dimethyl ethane (DME) as a solvent. Molecular Sieve (4 Å) was used as water scavenging agent in Example 2.

In particular, N,N-Dimethyl Laurylamide in dimethoxy ethane (DME), 5 mL in total, was introduced in the reactor, followed by the addition of 0.15 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard. The filtered samples were analyzed by gas chromatography.

The conversion, yield and selectivity were summarized in Table 1.

### Example (Ex.) 7: Hydrogenation of N,N-Dimethyl Laurylamide

Hydrogenation was performed in a 30 mL Taiatsu autoclave at 130° C. and 30 bar H₂ pressure at a substrate concentration of 100 wt. % for 17 hours in the presence of a V_{y}Oₓ/Rh/Al₂O₃ type catalyst prepared in Example 1.

N,N-dimethyl laurylamide was introduced in the reactor, followed by the addition of 0.15 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard.

The filtered samples were analyzed by gas chromatography.

The conversion, yield and selectivity were summarized in Table 1.

TABLE 1

| | | Rh-to- | | | | | | |
|---|---|---|---|---|---|---|---|---|
| | Amide | amide | Rh/V | Molecular | | | | |
| | Conc. | mol ratio | molar | Sieve | Reaction | Conv. | Yield | Select. |
| No. | (wt %) | (%) | ratio | (Y/N) | time (h) | (%) | (%) | (%) |
| Comp. 1 | 2.5 | 29 | 1/0 | Y | 1 | 8 | 5 | 57 |
| Ex. 2 | 2.5 | 29 | 1/1 | Y | 1 | 90 | 90 | 100 |
| Ex. 3 | 2.5 | 29 | 1/1 | N | 1 | 100 | 98 | 98 |
| Ex. 4 | 50 | 0.8 | 1/1 | N | 1 | 25 | 25 | 99 |
| Ex. 5 | 50 | 0.8 | 1/0.5 | N | 1 | 28 | 27 | 96 |
| Ex. 6 | 50 | 0.8 | 1/0.5 | N | 6 | 37 | 36 | 96 |
| Ex. 7 | 100 | 0.8 | 1/1 | N | 17 | 48 | 41 | 85 |

As can be seen in Table 1, the presence of vanadium on Rh/Al₂O₃ catalyst has a remarkable influence on the efficiency of the catalytic hydrogenation of N,N-dimethyl laurylamide into the respective amine. Whereas the unmodified catalyst in Comparative Example 1 achieved only 8% conversion of the amide after 1 hour reaction, the supported heterogeneous catalyst obtained in Example 1 was able to achieve 90% conversion and 100% selectivity during the same period of time.

In addition, the comparison of Example 2 and Example 3 showed that the utilization of molecular sieve as water scavenging agent in Example 2 does not influence the catalytic performance of the supported heterogeneous catalyst.

It can also be seen that, even without using DME as a solvent for the reaction (neat reaction conditions, Example 7), the supported heterogeneous catalysts can convert up to 48% of the aliphatic amide with an small drop in the selectivity of the process towards the amine (85%), after 17 hours. The drop in selectivity is mainly due to the formation of the secondary amine and dodecanol as side products. Nevertheless, such catalytic performance was obtained by using 0.8 mol % of rhodium as a function of the amide, which is significantly lower quantity of metal compared with the reactions performed under diluted conditions.

In addition, the comparison of Example 4 and Example 5 showed that by decreasing the amount of vanadium in the supported heterogeneous catalyst (Rh/V ratio from 1/1 to 1/0.5) the catalytic activity was kept intact.

### Example 8: Hydrogenation of N,N-Dimethyl Laurylamide

Catalyst was prepared as described in Example (Ex.) 1. The Rh-to-V molar ratio was kept at 1.0/0.5.

H₂-TPR and CO-chemisorption analysis of the prepared catalyst were performed in a Micromeritics AutoChem II 2920 apparatus with a thermal conductivity detector (TCD). For each experiment, approximately 100 mg catalyst was placed in a U-shaped quartz tube (i.d.=10 mm) and reduced in a flow of 10% H₂/Ar while ramping the temperature up to 200° C. at the rate of 10° C./min, and then held at 200° C. for 30 min (this part corresponds to the H₂-TPR analysis). After that, the sample was cooled down to 50° C. and flushed with He for 30 min. The loop gas of 10% CO/He was pulsed over the sample and the TCD signal was recorded until the peak area became constant (this part corresponds to the CO-TPD analysis).

The H₂-TPR profile and the CO-chemisorption result obtained for the prepared catalyst obtained (marked as “Acetylacetonate”) were shown in FIG. 1 and FIG. 2, respectively.

The catalytic performance was evaluated by using the reaction conditions described in Ex. 5, table 1, but running the reaction 5 hours instead of 1 hour.

In particular, N,N-Dimethyl Laurylamide in dimethoxy ethane (DME), 5 mL in total, was introduced in the reactor, followed by the addition of 0.15 g of catalyst. After closing the reactor, the system was purged at least 5 times with pure hydrogen, and then, pressurized at 30 bar H₂ pressure. Finally, the autoclave was placed inside a heated aluminum block, preheated at 130° C. After finishing the reaction, the reactor was cooled down with water, depressurized and opened to immediately add 1 mL of n-dodecane as internal standard. The filtered samples were analyzed by gas chromatography.

The conversion, yield and selectivity were summarized in Table 2.

### Example 9: Hydrogenation of N,N-Dimethyl Laurylamide

Catalyst was prepared as described in Example (Ex.) 1, but using ammonium metavanadate (NH₄VO₃) as vanadium precursor, and water as solvent for the impregnation. Rh-to-V molar ratio was kept at 1.0/0.5.

H₂-TPR and CO-chemisorption analysis of the prepared catalyst were performed with the same conditions as demonstrated in Example 8.

The H₂-TPR profile and the CO-chemisorption result obtained for the prepared catalyst (marked as “Vanadate”) were shown in FIG. 1 and FIG. 2, respectively.

It can be seen from FIG. 1 and FIG. 2, the vanadium precursor didn't have a strong influence neither on the H₂-TPR profile nor the CO uptake capabilities of the materials (Vanadate precursor vs Acetylacetonate precursor).

The catalytic performance was evaluated by using the reaction conditions described in Ex. 5, Table 1, but running the reaction 5 hours instead of 1 hour.

In particular, N,N-Dimethyl Laurylamide in dimethoxy ethane (DME), 5 mL in total, was introduced in the reactor, followed by the addition of 0.15 g of catalyst. After closing the reactor, the system was purged at least 5 times with pure hydrogen, and then, pressurized at 30 bar H₂ pressure. Finally, the autoclave was placed inside a heated aluminum block, preheated at 130° C. After finishing the reaction, the reactor was cooled down with water, depressurized and opened to immediately add 1 mL of n-dodecane as internal standard. The filtered samples were analyzed by gas chromatography.

The conversion, yield and selectivity were summarized in Table 2.

### Comparative Example 2: Hydrogenation of N,N-Dimethyl Laurylamide

A Rh/V co-impregnated catalyst was prepared by wet impregnation of γ-Al₂O₃ support, as described in Example (Ex) 1, but using a solution composed by vanadyl acetylacetonate and rhodium acetylacetonate in acetone. The Rh metal loading was kept at 5 wt %, relative to the total weight of the catalyst, having a Rh-to-V molar ratio of 1.0/0.5.

H₂-TPR and CO-chemisorption analysis of the prepared catalyst were performed with the same conditions as demonstrated in Example 8.

The H₂-TPR profile and the CO-chemisorption result obtained for the prepared catalyst (marked as “Rh/V co-impreg.”) were shown in FIG. 1 and FIG. 2, respectively.

It can be seen from FIG. 1 and FIG. 2 that the catalyst prepared by co-impregnation didn't show any reduction peak below 200° C., which indicates that the co-impregnation methodology provokes the formation of a Rh—V mixed oxide (after calcination at 300° C.), which is difficult to be reduced under the used reaction conditions (130° C., 30 bar H₂).

In addition, the CO uptake observed on the catalyst prepared by co-impregnation was smaller compared to the catalyst prepared by sequential impregnation method (in Examples 8 and 9), indicating that less Rh is available for the chemisorption process. It can be concluded that the sequential impregnation method facilitates the co-existence of metallic rhodium and oxidized vanadium species as active phases for the catalytic hydrogenation of amides. However, in the case of the co-impregnation method, the formation of Rh—V-type solid solutions makes difficult the reducibility of Rh, thus affecting the catalytic performance of such system.

The catalytic performance was evaluated by using the reaction conditions described in Ex. 5, Table 1, but running the reaction 5 hours instead of 1 hour.

In particular, N,N-Dimethyl Laurylamide in dimethoxy ethane (DME), 5 mL in total, was introduced in the reactor, followed by the addition of 0.15 g of catalyst. After closing the reactor, the system was purged at least 5 times with pure hydrogen, and then, pressurized at 30 bar H₂ pressure. Finally, the autoclave was placed inside a heated aluminum block, preheated at 130° C. After finishing the reaction, the reactor was cooled down with water, depressurized and opened to immediately add 1 mL of n-dodecane as internal standard. The filtered samples were analyzed by gas chromatography.

The conversion, yield and selectivity were summarized in Table 2.

TABLE 2

| | Vanadium | Rhodium | Synthesis | Conv. | Yield | Select. |
|---|---|---|---|---|---|---|
| No. | precursor | precursor | method | (%) | (%) | (%) |
| Ex. 8 | V(Acac)₂ | Rh/Al₂O₃ | Impreg. | 41 | 39 | 95 |
| Ex. 9 | NH₄VO₃ | Rh/Al₂O₃ | Impreg. | 38 | 35 | 92 |
| Comp. | V(Acac)₂ | Rh/Al₂O₃ | Co-impreg. | 32 | 31 | 97 |
| 2 | | | | | | |

It can be seen from Table 2 that, regardless the vanadium precursor used for the catalyst's synthesis (i.e. V(Acac)₂ vs NH₄VO₃) a very similar catalytic performance can be achieved, being slightly better the catalyst prepared with the organometallic precursor. Nevertheless, in the catalyst prepared by co-impregnation, it is clear a drop in the catalytic efficiency.

### Examples 10-12: Hydrogenation of N,N-Dimethyl Laurylamide

Hydrogenation was performed in a Top-Industry reaction system at 40 bar H₂ pressure at a given temperature as specified in Table 3 in the presence of 1.0 g V_{y}Oₓ/Rh/Al₂O₃ type catalyst with Rh/V molar ratio of 1/1 prepared in Example 1 for 1 hour. The hydrogenation was carried out under diluted condition using 50 ml dimethyl ethane (DME) as a solvent for 5.0 mmol N,N-dimethyl laurylamide. 1.0 g molecular Sieve (4 Å) was used as water scavenging agent.

In particular, N,N-dimethyl laurylamide in dimethoxy ethane (DME) was introduced in the reactor, followed by the addition of 0.15 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard. The filtered samples were analyzed by gas chromatography.

The conversion, yield and selectivity were summarized in Table 3.

TABLE 3

| | | Rh/V | Molecular | | | | |
|---|---|---|---|---|---|---|---|
| | | molar | Sieve | Reaction | Conv. | Yield | Select. |
| No. | Temperature(° C.) | ratio | (Y/N) | time (h) | (%) | (%) | (%) |
| Ex. 10 | 100 | 1/1 | Y | 1 | 93 | 93 | 100 |
| Ex. 11 | 110 | 1/1 | Y | 1 | 98 | 98 | 100 |
| Ex. 12 | 120 | 1/1 | Y | 1 | 99 | 99 | 100 |

It can be seen that the conversion was above 90% and the selectivity can reach 100% at a mild hydrogenation condition with a temperature of 100-120° C. and a hydrogen pressure of 40 bar under diluted condition.

### Examples 13-14: Hydrogenation of N,N-Dimethyl Laurylamide

Hydrogenation was performed in a Top-Industry reaction system at 110° C. at a given H₂ pressure as specified in Table 4 in the presence of 1.0 g V_{y}Oₓ/Rh/Al₂O₃ type catalyst with Rh/V molar ratio of 1/1 prepared in Example 1 for 1 hour. The hydrogenation was carried out under diluted condition using 50 ml dimethyl ethane (DME) as a solvent for 5.0 mmol N,N-dimethyl laurylamide. 1.0 g molecular Sieve (4 Å) was used as water scavenging agent.

In particular, N,N-dimethyl laurylamide in dimethoxy ethane (DME) was introduced in the reactor, followed by the addition of 0.15 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard. The filtered samples were analyzed by gas chromatography.

The conversion, yield and selectivity were summarized in Table 4.

TABLE 4

| | Pressure | Rh/V molar | Molecular | Reaction | Conv. | Yield | Select. |
|---|---|---|---|---|---|---|---|
| No. | (bar) | ratio | Sieve (Y/N) | time (h) | (%) | (%) | (%) |
| Ex. 13 | 30 | 1/1 | Y | 1 | 92 | 92 | 100 |
| Ex. 14 | 35 | 1/1 | Y | 1 | 97 | 97 | 100 |
| Ex. 11 | 40 | 1/1 | Y | 1 | 98 | 98 | 100 |

It can be seen that the conversion was above 90% and the selectivity can reach 100% at a mild hydrogenation condition with a temperature of 110° C. and a hydrogen pressure of 30-40 bar under diluted condition.

### Examples 15-18: Hydrogenation of N,N-Dimethyl Laurylamide

Hydrogenation was performed in a Taiatsu autoclave at 130° C. and 50 bar H₂ pressure at a substrate concentration of 100 wt. % for given time, as specified in Table 5 in the presence of the V_{y}Oₓ/Rh/Al₂O₃ type catalyst with Rh/V molar ratio of 1/1 prepared in Example 1. 5 ml N,N-dimethyl laurylamide was used.

In particular, N,N-dimethyl laurylamide was introduced in the reactor, followed by the addition of 0.15 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard.

The conversion, yield and selectivity were summarized in Table 5.

TABLE 5

| | Catalyst | Mol. Sieve | Pressure | Time | Conversion | Yield | Select. |
|---|---|---|---|---|---|---|---|
| No. | (g) | (g) | (bar) | (h) | (%) | (%) | (%) |
| Ex. 15 | 0.1 | 0.3 | 50 | 1 | 11 | 11 | 100 |
| Ex. 16 | 0.3 | 0.1 | 50 | 1 | 26 | 25 | 96 |
| Ex. 17 | 0.3 | 0.1 | 50 | 17 | 59 | 53 | 90 |
| Ex. 18 | 0.6 | 0.2 | 50 | 17 | 67 | 59 | 88 |

### Example 19: Hydrogenation of N,N-Dimethyl Laurylamide

The robustness and reusability of the V_{y}Oₓ/Rh/Al₂O₃ type catalyst with Rh/V molar ratio of 1/1 prepared in Example 1 was evaluated under diluted.

The reaction was performed in a Top-industry reaction system.

Reaction conditions were as follows:

T=130° C.,

P_{H2}=30 bar,

Reaction time t=1 h,

N,N-dimethyl lauryl amide=0.5 mmol,

DME (solvent)=5 mL,

Rh:V(1:1)/Al₂O₃ catalyst=0.3 g.

The catalyst was recovered by centrifugation after every catalytic cycle, washed with ethanol and DME, and then, used for the next reaction. The conversion and selectivity were summarized in Table 6 below.

TABLE 6

| Catalytic run | Conv. (%) | Select. (%) |
|---|---|---|
| 1^{st} | 100 | 98 |
| 2^{nd} | 100 | 98 |
| 3^{rd} | 100 | 98 |
| 4^{th} | 97 | 100 |
| 5^{th} | 92 | 100 |
| 6^{th} | 89 | 100 |

It can be seen from Table 6 that under diluted conditions, neither activity nor selectivity was significantly affected by re-using the catalysts up to 6 times.

Inductively coupled plasma atomic emission spectroscopy (ICP-AES) confirmed the absence of rhodium and/or vanadium in the final reaction mixture (liquid phase).

### Example 20: Hydrogenation of N,N-Dimethyl Laurylamide

The robustness and reusability of the V_{y}Oₓ/Rh/Al₂O₃ type catalyst with Rh/V molar ratio of 1/1 prepared in Example 1 was evaluated under neat reaction conditions.

The reaction was performed in a Taiatsu Autoclave.

Reaction conditions were as follows:

T=130° C.,

P_{H2}=30 bar,

Reaction time t=17 h,

N,N-dimethyl lauryl amide=0.5 mmol,

No solvent,

Rh:V(1:1)/Al₂O₃ catalyst=0.3 g.

The catalyst was recovered by centrifugation after every catalytic cycle, washed with ethanol and DME, and then, used for the next reaction. The conversion and selectivity were summarized in Table 7 below.

TABLE 7

| Catalytic run | Conv. (%) | Select. (%) |
|---|---|---|
| 1^{st} | 48 | 85 |
| 2^{nd} | 47 | 91 |
| 3^{rd} | 47 | 91 |
| 4^{th} | 32 | 96 |

It can be seen from Table 7 that under neat reaction conditions, although there was not significant drop in the catalytic activity after 3-times reuse, the 4^{th} reuse shown certain deterioration in conversion but not in selectivity. This fact could be related to loss of catalysts after every reuse.

### Example 21: Hydrogenation of Lauramide

The V_{y}Oₓ/Rh/Al₂O₃ type catalyst with Rh/V molar ratio of 1/1 prepared in Example 1 was used for the hydrogenation of lauramide (primary amide).

In particular, lauramide in dimethoxy ethane (DME) was introduced in the reactor, followed by the addition of 0.1 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard. The filtered samples were analyzed by gas chromatography.

The preliminary results show a conversion of 60% and a selectivity of 65% under diluted condition.

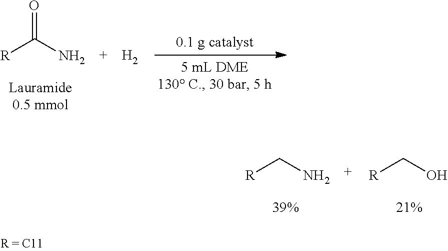

### Example 22: Hydrogenation of Benzamide

The V_{y}Oₓ/Rh/Al₂O₃ type catalyst with Rh/V molar ratio of 1/1 prepared in Example 1 was used for the hydrogenation of benzamide (primary amide).

In particular, benzamide in dimethoxy ethane (DME) was introduced in the reactor, followed by the addition of 0.1 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard. The filtered samples were analyzed by gas chromatography.

The preliminary results show a conversion of 100% and a selectivity of 70% under diluted condition.

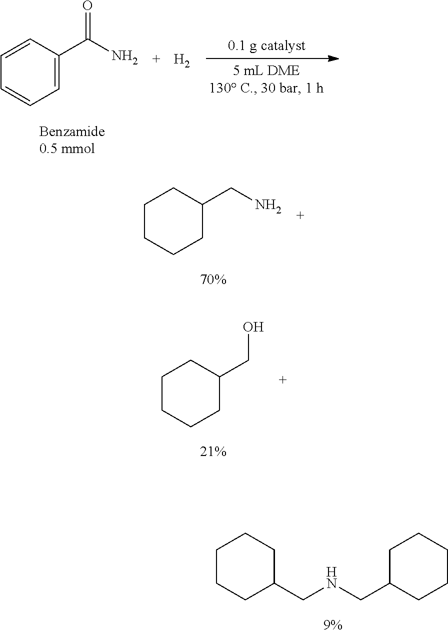 [SMILES: NC(=O)c1ccccc1 ; "H2" ; NCC1CCCCC1 ; OCC1CCCCC1 ; C1CCC(CNCC2CCCCC2)CC1]

### Comparative Examples 3-4 and Examples 23-24: Hydrogenation of 1-acetyl Piperidine

Hydrogenation of 1-acetyl piperidine in the presence of different catalysts was evaluated as follows.

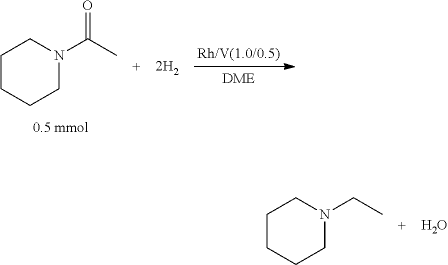

Hydrogenation of 1-acetyl piperidine was performed in a Taiatsu Autoclave reaction system at a temperature of 70° C. or 130° C. and a hydrogen pressure of 10 bar or 30 bar at a given substrate concentration for a reaction time of 1 hour or 16 hours in the presence of a catalyst listed in Table 8. The hydrogenation was carried out under diluted condition using 5 mL dimethyl ethane (DME) as a solvent for 0.5 mmol 1-acetyl piperidine.

In comparative Examples 3 and 4, the catalysts used were prepared by co-impregnation. In Examples 22 and 23, the catalysts used were prepared according to the procedure in Example 1.

For all catalysts used, the molar ratio of Rh/V is 1.0:0.5, with the amount of Rh is 5 wt. %, relative to the total weight of the catalyst used.

In particular, 1-acetyl piperidine in dimethoxy ethane (DME) was introduced in the reactor, followed by the addition of 0.1 g of catalyst. After closing the reactor, the system was purged at least 5 times with pure hydrogen, and then, pressurized at the desired H₂ pressure. Finally, the autoclave was placed inside a heated aluminum block, preheated at the given reaction temperature. After finishing the reaction, the reactor was cooled down with water, depressurized and opened to immediately add 1 mL of n-dodecane as internal standard. The filtered samples were analyzed by gas chromatography.

The yield was summarized in Table 8.

TABLE 8

| No. | Catalyst | T (° C.) | P_{H2} (bar) | Time (h) | Yield (%) |
|---|---|---|---|---|---|
| Comp. 3 | Rh/V/HAP | 70 | 30 | 1 | 6 |
| Comp. 4 | Rh/V/SiO₂ | 130 | 10 | 16 | 50 |
| Ex. 23 | Rh/V/Al₂O₃ | 70 | 30 | 1 | 45 |
| Ex. 24 | Rh/V/Al₂O₃ | 130 | 10 | 1 | 85 |

It can be seen from Table 8 that as compared with the process employing Rh/V-based catalysts prepared by co-impregnation, the process according to the present invention performs much better under similar reaction conditions.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A process for converting an amide into an amine comprising hydrogenation of the amide at a temperature not higher than 130° C. and a hydrogen pressure not higher than 50 bar in the presence of a supported heterogeneous catalyst preparable by a method comprising depositing vanadium on a supported noble metal catalyst by impregnation.

2. The process according to claim 1, wherein the amide is of formula (I) and the amine is of formula (II), 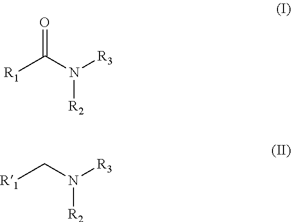
wherein
R₁ is a group selected from linear or branched C1-C20 alkyl, and phenyl which is optionally substituted by a linear or branched C1-C4 alkyl,
R′₁ is identical to R1 when R1 is a linear or branched C1-C20 alkyl and R′1 is cyclohexyl which is optionally substituted by a linear or branched C1-C4 alkyl when R1 is phenyl which is optionally substituted by a linear or branched C1-C4 alkyl,
R₂ and R₃, independently from each other, are hydrogen, or linear or branched C1-C4 alkyl, or
R₂ and R₃ together with the nitrogen atom they attached to form a piperidine ring which is optionally substituted by a linear or branched C1-C4 alkyl.

3. (canceled)

4. (canceled)

5. The process according to claim 1, wherein the noble metal is selected from the group consisting of rhodium(Rh), platinum (Pt), ruthenium (Ru), and iridium (Ir).

6. The process according to claim 1, wherein the noble metal is present in amount from 1 wt. % to 10 wt. % in the supported heterogeneous catalyst, relative to the total weight of the supported heterogeneous catalyst.

7. The process according to claim 1, wherein vanadium is present in amount from 0.5 wt. % to 10 wt. % in the supported heterogeneous catalyst, relative to the total weight of the supported heterogeneous catalyst.

8. The process according to claim 1, wherein the molar ratio of the noble metal to vanadium is from 0.5 to 10.

9. The process according to claim 1, wherein the support for the supported heterogeneous catalyst is selected from alumina, silica and activated carbon.

10. The process according to claim 1, wherein the hydrogenation is carried out with the molar ratio of the noble metal in the supported heterogeneous catalyst to the amide from 0.5% to 35%.

11. A process for converting an amide into an amine comprising:
(i) preparing a supported heterogeneous catalyst by a method comprising depositing vanadium on a supported noble metal catalyst by impregnation, and
(ii) causing hydrogenation of the amide at a temperature not higher than 130° C. and a hydrogen pressure not higher than 50 bar in the presence of the so-prepared supported heterogeneous catalyst to obtain the amine.

12. The process according to claim 3, wherein the amide is of formula (I) and the amine is of formula (II), 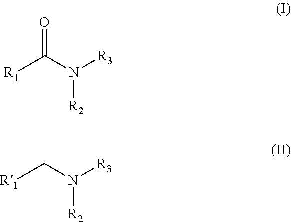
wherein
R₁ is a group selected from linear or branched C1-C20 alkyl, and phenyl which is optionally substituted by a linear or branched C1-C4 alkyl,
R′₁ is identical to R₁ when R₁ is a linear or branched C1-C20 alkyl and R′₁ is cyclohexyl which is optionally substituted by a linear or branched C1-C4 alkyl when R1 is phenyl which is optionally substituted by a linear or branched C1-C4 alkyl,
R₂ and R₃, independently from each other, are hydrogen, or linear or branched C1-C4 alkyl, or
R₂ and R₃ together with the nitrogen atom they attached to form a piperidine ring which is optionally substituted by a linear or branched C1-C4 alkyl.

13. The process according to claim 11, wherein depositing vanadium on the supported noble metal catalyst comprises depositing a vanadium precursor on the supported noble metal catalyst by impregnation.

14. The process according to claim 13, wherein the vanadium precursor is selected from the group consisting of Vanadyl (IV) acetylacetonate and ammonium metavanadate.

15. The process according to claim 11, wherein depositing vanadium on a supported noble metal catalyst is carried out as follows:
i) dissolving a vanadium precursor in a solvent to obtain a vanadium-containing solution,
ii) adding the supported noble metal catalyst to the vanadium-containing solution to form an uniform mixture;
iii) evaporating the solvent to yield a powder;
iv) drying and calcining the powder to obtain the supported heterogeneous catalyst.

16. The process according to claim 15, wherein the solvent is selected from the group consisting of acetone, water, and ethanol.

17. (canceled)

18. (canceled)

19. (canceled)

20. (canceled)

21. (canceled)

22. (canceled)

23. (canceled)

24. (canceled)

25. (canceled)

26. A mixture comprising a first amine of formula (II) and an alcohol of formula (III): 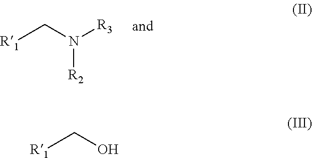
wherein:
R′₁ is cyclohexyl which is optionally substituted by a linear or branched C1-C4 alkyl;
R₂ and R₃, independently from each other, are hydrogen, or linear or branched C1-C4 alkyl;
the molar ratio of the first amine to the alcohol is greater than 2.5.

27. The mixture according to claim 5, wherein R₃ is H.

28. The mixture according to claim 26, further comprising a second amine of formula (IV): 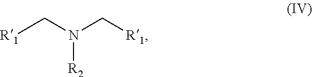
wherein R′₁ is cyclohexyl which is optionally substituted by a linear or branched C1-C4 alkyl; and
R₂ is hydrogen, or linear or branched C1-C4 alkyl.

29. The mixture according to claim 26, wherein R₂ is H.

30. The mixture according to claim 28, wherein the molar ratio of the first amine to the second amine is greater than 5.

31. The mixture according to any one of claim 26, wherein R′₁ is cyclohexyl.
